(19) United States
(12) Patent Application Publication
(10) Pub. No.: US 2014/0031437 A1
(43) Pub. Date: Jan. 30, 2014

(54) OIL-IN-WATER EMULSIONS

(71) Applicants: Christopher Smith, Mansfield (GB); Giovanni Francesco Unali, Bebington (GB)

(72) Inventors: Christopher Smith, Mansfield (GB); Giovanni Francesco Unali, Bebington (GB)

(21) Appl. No.: 14/009,258

(22) PCT Filed: Mar. 2, 2012
(86) PCT No.: PCT/EP12/53611

§ 371 Date: Oct. 1, 2013

(30) Foreign Application Priority Data
Apr. 12, 2011 (EP) 11162004.3

(51) Int. Cl. A61K 8/73; A61Q 19/00; A61K 8/92

(52) U.S. Cl. CPC A61K 8/731; A61K 8/92; A61Q 19/00
USPC 514/781

(57) ABSTRACT

The invention provides an oil-in-water emulsion suitable for cosmetic or personal care use, the emulsion comprising: a) a aqueous continuous phase; b) a dispersed oil phase, and c) optionally, a nonionic emulsifier; in which the aqueous continuous phase is structured by a dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols; and in which the emulsion comprises less than 0.2 wt % anionic surfactant (by total weight anionic surfactant based on the total weight of the emulsion).

This invention relates to stabilised oil-in-water emulsions comprising modified cellulose.

### BACKGROUND

Oil-in-water emulsions are widely employed in the cosmetic and dermatological fields for various reasons such as their ease of use and skin conditioning properties.

In order to form stable oil-in-water emulsions, a hydrocolloid is used to provide a supporting structure. Suitable polymers tend to be synthetic, expensive or both. There are some natural polymers offering such attributes but they can be difficult to process, have poor compatibility profiles or often leave a sticky skin feel.

Cellulose is a natural, plentiful, and consequently inexpensive, biopolymer. However, in its unmodified form it is completely insoluble and cannot be dispersed into an aqueous liquid composition to achieve a stable, thickened, product.

Partially and selectively oxidising cellulose at the C6 position creates cellouronates or cellouronic acids which are more water dispersible than cellulose but still relatively insoluble.

WO 2010/076292 describes how this type of oxidised cellulose may be used as an alternative structurant for aqueous detergent compositions. However, WO 2010/076292 emphasises that in order to provide gelled material it is essential to use anionic or zwitterionic surfactants, and describes a gelled emulsion of liquid paraffin or silicone oil requiring 5.7% SLES 1EO.

Surprisingly, we have now found that the oxidised cellulose described in WO2010/076292 is capable of stabilising oil-in-water emulsions in the absence of any anionic or zwitterionic surfactant. Furthermore, the resulting emulsions have excellent stability, appearance and sensory feel.

### SUMMARY OF THE INVENTION

Accordingly the present invention provides an oil-in-water emulsion suitable for cosmetic or personal care use, the emulsion comprising:

a) a aqueous continuous phase;
b) a dispersed oil phase, and
c) optionally, a nonionic emulsifier;
in which the aqueous continuous phase is structured by a dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
and in which the emulsion comprises less than
0.2 wt % anionic surfactant (by total weight anionic surfactant based on the total weight of the emulsion).

### DETAILED DESCRIPTION OF THE INVENTION

### Aqueous Continuous Phase

The oil-in-water emulsion of the present invention comprises a aqueous continuous phase.

The amount of water in the emulsion of the invention generally ranges from 50 to 90 wt %, and preferably ranges from 70 to 85 wt % (by weight water based on the total weight of the emulsion).

### Modified Cellulose Biopolymer

The oil-in-water emulsion of the present invention comprises a dispersed modified cellulose biopolymer which serves to provide structure to the aqueous continuous phase.

The amount of modified cellulose biopolymer in the emulsion of the invention generally ranges from 0.5 to 5 wt %, and preferably ranges from 1 to 2 wt % (by total weight modified cellulose biopolymer based on the total weight of the emulsion).

The modified cellulose biopolymer for use in the invention may be characterised as a water insoluble, water dispersible modified cellulose in which only a proportion of its C6 primary alcohol groups have been oxidised to acid groups. Cellulose where all such alcohols have been oxidised is called polyuronic acid or polyglucuronic acid. Such fully oxidised material is soluble in water. It is unsuitable for use in the present invention for two reasons. Firstly, the cost of the extra processing required to create more than 70% substitution of primary alcohols by carboxylic acid groups makes it not cost effective as a replacement for surfactant and second the highly oxidised material tends to include unwanted depolymerised cellulose, which leads to a reduction of yield of insoluble dispersible structurant.

In the context of the present invention, a modified cellulose biopolymer is said to be water soluble, if it leaves less than 10 wt % of its dry mass as undissolved residue when a 2 g dry sample is added to 1 litre of agitated demineralised water at 25° C.

Totally unoxidised (unmodified) cellulose is unable to function as a structurant. Oxidising the cellulose to have at least 10% of the primary alcohols converted to carboxylic acids makes the cellulose dispersible in water and when mixed within the surfactant system the resulting structured liquid or gel maintains the cellulose in a dispersed state so it does not settle over time.

### The Cellulose Starting Material

Several factors influence the choice of a suitable starting material.

More porous unmodified cellulosic material will oxidise more rapidly. Characterisation of surface area or porosity is readily achieved by porosimetry or BET measurements. In general, those starting materials that oxidise more rapidly due to their low crystallinity and higher surface area and/or porosity, prove easier to disperse than those that oxidise less rapidly.

The rate of oxidation is also affected by the dimensions of the particles of cellulose starting material; the reduction in rate for longer (>500 micron) fibres is significant. Fibres less than 500 microns long are therefore preferred for this reason and due to the added difficulty in agitation of the longer fibres. While oxidation results in significant gross particle size reduction, this does not compensate for decreased fibril surface accessibility in the long fibres.

Celluloses that have not been previously subjected to acid hydrolysis are a preferred starting material, due to reactivity, cost and resultant product dispersibility.

Relatively unrefined α-cellulose, for example filter aid fibres, provides one of the most readily oxidised and dispersed sources of cellulose. Advantageously, the oxidation process also serves to bleach coloured components, such as lignin, in such unbleached cellulose starting materials. This then renders such materials more suitable for use in contexts where visual clarity of the end product is desirable, for example transparent personal care formulations.

### Oxidation

Because of its known specificity for primary alcohol oxidation TEMPO-mediated oxidation of cellulose is preferred (i.e. 2,2,6,6-tetramethylpiperidine-1-oxyl and related nitroxy radical species). The process proceeds well without cooling, at relatively high weight % cellulose in the initial suspension. Simple workup procedures afford clean material suitable for dispersion. Such TEMPO mediated oxidation of cellulose is described in the published literature and the skilled worker will be able as a matter of routine to adapt known methods to achieve the oxidation required by this invention.

While aqueous NaOCl/TEMPO/NaBr is a highly preferred oxidation system, there are a number of other systems available to the skilled worker, especially for large scale production. Among such systems, there may be mentioned use of peracetic acid or monoperoxysulfate salts (Oxone®) as the oxidant with 4-acetamido-2,2,6,6-tetramethylpiperidine-1-oxyl (4-acetamido-TEMPO) as the radical transfer catalyst or mediator and sodium bromide co-catalyst for the oxidation. Elimination of chlorine from the oxidation system is environmentally desirable.

The use of 4-acetamido-TEMPO as radical transfer catalyst is also advantageous as, although it has a higher molecular weight than TEMPO, it has significantly lower vapour pressure reducing potential exposure hazards. Many other 4-substituted TEMPO analogues exist, but many, such as 4-hydroxy-TEMPO exhibit poor stability. TEMPO on solid supports or on soluble polymers may be used.

Electrochemical oxidation is a potentially clean means of effecting oxidation of carbohydrate moieties, although mediation by a radical transfer catalyst (such as TEMPO) is still required.

Laccase mediated oxidation, which also requires a radical transfer catalyst (e.g. TEMPO) but replaces the oxidant with an enzyme, may advantageously be used.

Using the TEMPO system the degree of reproducibility of oxidation of cellulose from the same source is good.

### Degree of Oxidation

In the context of the present invention, the term “degree of oxidation” of the modified cellulose means the percentage glucose units oxidised to carboxylic acid as measured by titration with sodium hydroxide. It is assumed that all oxidation takes place at the primary alcohol positions. A reasonable assumption, given that primary alcohol specific oxidation chemistry is employed. Furthermore it is assumed that all oxidation leads to carboxylic acid formation.

Degree of polymerisation (DP) does not seem greatly to influence the performance of the modified cellulose. The key thing is that the modified cellulose must remain insoluble.

During oxidation, there is some degradation of the cellulose allowing release of polymer chains. It is particularly advantageous to keep this to a minimum in order to increase the yield of the modified insoluble cellulose material suitable for structuring applications. We have determined that above 70% oxidisation, the yield is unacceptably low and the processing costs become unacceptably high.

The degree of oxidation of the modified cellulose lies in the range 10 to 70%. As the degree of oxidation increases, the amount of soluble material produced will rise and this reduces the yield of insoluble structuring material, thus the higher degrees of oxidation confer no real structuring benefits. For this reason, it is preferred to restrict the degree of oxidation to 60%, or even 50% and the most preferred modified materials have degrees of oxidation even lower than 40% or sometimes even lower than 30%.

To achieve a high enough dispersibility/solubility for the modified cellulose to act as a structurant it must be oxidised to at least 10%. The exact amount of oxidation required for a minimum effect will vary according to the starting material used. Preferably, it is at least 15% oxidised and most preferably, at least 20% oxidised.

### Dispersal of the Modified Cellulose

At small scale, high energy sonication is the preferred method to give the high shear necessary to achieve the aqueous dispersion of the modified cellulose. However, other techniques are more suitable for large scale applications. These include the use of a high speed and high shear stirrer, or a blender, or a homogeniser. Homogenisation may achieve higher levels of dispersed material than are attainable via sonication.

When degrees of oxidation of less than 10% are used, the partially oxidised cellulose proves too resistant to dispersion to produce a transparent or translucent mixture and higher energy input is required. Provided the lower limit of 10% is exceeded, those modified celluloses with a lesser degree of oxidation appear to provide greater structuring capacity once dispersed. This is attributed to less degradation of the material during oxidation and thus the existence of longer individual dispersed (not dissolved) fibrils. This may be because the structure of the cellulose starting material is partially retained, but the fibrils are rendered dispersible by the introduction of negatively charged functional groups on the surface during oxidation.

Oxidised, dispersed cellulose is a largely insoluble polymer that occurs in the form of well dispersed fibrils rather than isolated solvated polymer chains. The fibrils have a large aspect ratio and are thin enough to provide almost transparent dispersions. Carboxylate groups provide anionic surface charge, which results in a degree of repulsion between fibrils, militating against their reassociation into larger structures. Addition of acid to dispersions of oxidised cellulose results in separation of gelled material while at pH between ca 5-9 fibrils may be maintained in a dispersed form as the COO— salt of an appropriate counterion.

This allows the formulator to make a stock of aqueous dispersion of the modified cellulose, with remaining process steps carried out and further ingredients added as and when necessary to enable easy late-stage variations in composition before products are packaged.

### Nonionic Emulsifier

Preferably the oil-in-water emulsion of the present invention comprises a nonionic emulsifier.

Typically such a nonionic emulsifier is an oil-in-water (O/W) emulsifier having an HLB (Hydrophile-Lipophile Balance) value ranging from 8 to 18.

Suitable nonionic emulsifiers of this type may be selected from alkoxylate emulsifiers.

The term “alkoxylate emulsifier” as used herein generally means surfactants in which a hydrophobe, usually a hydrocarbyl group, is connected through the residue of a linking group having a reactive hydrogen atom to an oligomeric or polymeric chain of alkylene oxide residues. The hydrocarbyl group is typically a chain, commonly an alkyl or alkenyl chain, containing from 8 to 24, particularly 12 to 22, and usually 14 to 20 carbon atoms. The linking group can be an oxygen atom (hydroxyl group residue); a carboxyl group (fatty acid or ester residue); an amino group (amine group residue); or a carboxyamido (carboxylic amide residue). The alkylene oxide residues are typically residues of ethylene oxide (C₂H₄O) or propylene oxide (C₃H₆O) or combinations of ethylene and propylene oxide residues. When combinations are used the proportion of ethylene oxide residues will usually be at least about 50 mole % and more usually at least 75 mole %, the remainder being propylene oxide residues. Preferably substantially all the residues are ethylene oxide residues. The number of alkylene oxide residues is usually from 2 to 200 per mole of alkoxylate emulsifier. Examples of suitable alkoxylate emulsifiers include alcohol alkoxylates of the formula R¹—O-(AO)ₙ—H; fatty acid alkoxylates of the formula R¹—COO-(AO)ₙ—R²; fatty amine alkoxylates of the formula R¹—NR³-(AO)ₙ—H; and fatty amide alkoxylates of the formula R¹—NR³-(AO)ₙ—, —H, where each R¹ is independently a C₈ to C₂₄, preferably a C₁₂ to ₂₂ hydrocarbyl, preferably alkyl or alkenyl, group; R² is a hydrogen atom or a C₁ to C₆ alkyl group; and each R³ is independently a C₁ to C₆ alkyl group or a group (AO)ₙ—H; each AO is independently an ethylene oxide or propylene oxide group; and the total of the indices n in the molecule is from 2 to 200.

Alternatively, nonionic emulsifiers that are not derivatives of alkylene oxides can be used. This may be preferable where it is desired to formulate systems which are derived entirely from natural, especially vegetable, source materials.

Examples of nonionic emulsifiers that can be derived from natural materials include fatty acid esters, ethers, hem i-acetals or acetals of polyhydroxylic compounds or a fatty acid amide which is N-substituted with the residue of a polyhydroxylic compound.

Suitable esters of polyhydroxylic compounds include saccharide esters, and particularly mono- and/or diesters of fatty acids of formula R¹—COON (where R¹ is as defined above for alkoxylate emulsifiers) with a sugar, especially sucrose, fructose, glucose and/or alkylglucose (e.g. methylglucose or ethylglucose). Commercially available sugar esters are usually mixtures containing mono-ester, higher esters and sometimes free starting material (sugar). Examples include glucose palm itate, methylglucose isostearate, methylglucose laurate, methylglucose sesquistearate (mixture of the mono- and diesters), alkylglucose palmitates such as methylglucose or ethylglucose palmitate, methyl glucose dioleate, methyl glucose sesquiisostearate, sucrose palmitate, sucrose stearate and sucrose monolaurate.

Also suitable are polyglyceryl ethers of the above-described sugar esters, such as polyglyceryl-3 methylglucose distearate (a diester of stearic acid and the condensation product of methylglucose and polyglycerin-3).

Other suitable esters of polyhydroxylic compounds include esters of fatty acids, particularly fatty acids having from 8 to 24, preferably 12 to 22, more preferably 16 to 20 carbon atoms, and polyols, particularly glycerol, or a polyglycerol, or an anhydrosaccharide such as sorbitan. Examples include glyceryl monolaurate, glyceryl monooleate, glyceryl monolinoleate, glyceryl monostearate, glyceryl monoisostearate, glyceryl trioctanoate, glyceryl triisostearate, polyglyceryl-3 stearate, polyglyceryl-3 cocoate, sorbitan monooleate, sorbitan monostearate, sorbitan monolaurate, and sorbitan monopalmitate.

Suitable ethers of polyhydroxylic compounds include alkyl polysaccharides of the formula: R¹—O-(G)ₐ, where R¹ is as defined above for alkoxylate emulsifiers; each G is independently a saccharide residue, preferably a glucose residue and a is from 1 to about 5. Examples include decylglucoside, caprylyl/capryl glucoside, laurylglucoside, cocoglucoside, cetostearyl glucoside, arachidyl glucoside, and cocoylethylglucoside.

Another suitable type of naturally-derivable nonionic emulsifier includes fatty acid esters of hydroxycarboxylic acids, in which the fatty acid typically has from 8 to 24, preferably from 12 to 22, more preferably from 16 to 20 carbon atoms and the hydroxycarboxylic acid is preferably citric acid.

Another suitable type of naturally-derivable nonionic emulsifier includes N-substituted fatty acid amides in which the N-substituent is the residue of a polyhydroxylic compound, for example a saccharide residue such as a glucosyl group. This type of emulsifier typically has the formula: R¹—CO—NR⁵, R⁶, where R¹ is as defined above for alkoxylate emulsifiers; R⁵ is a hydrogen atom, a C₁ to C₆ alkyl group or a group of the formula R⁶; and R⁶ is a polyhydroxyl hydrocarbyl group, particularly a group containing from 3 to 10 carbon atoms and 2 to 6 hydroxyl groups, preferably a glucosyl residue.

Mixtures of any of the above described materials may also be used.

The amount of nonionic emulsifier in the emulsion of the invention generally ranges from 0 to 10 wt %, and preferably ranges from 1 to 5 wt % (by total weight nonionic emulsifier based on the total weight of the emulsion).

### Anionic Surfactant

The oil-in-water emulsion of the present invention comprises less than 0.2 wt % anionic surfactant (by total weight anionic surfactant based on the total weight of the emulsion). Preferably the emulsion is substantially free of anionic surfactant. The term “substantially free” in this particular context generally means that the emulsion comprises less than 0.1%, more preferably less than 0.01%, most preferably less than 0.001% by total weight anionic surfactant based on the total weight of the emulsion.

Examples of anionic surfactants include the sodium, magnesium, ammonium or ethanolamine salts of C₈ to C₁₈ alkyl sulphates (for example sodium dodecyl sulphate), C₈ to C₁₈ alkyl sulphosuccinates (for example dioctyl sodium sulphosuccinate), C₈ to C₁₈ alkyl sulphoacetates (such as sodium dodecyl sulphoacetate), C₈ to C₁₈ alkyl sarcosinates (such as sodium dodecyl sarcosinate), C₈ to C₁₈ alkyl phosphates (which can optionally comprise up to 10 ethylene oxide and/or propylene oxide units) and sulphated monoglycerides.

### Dispersed Oil Phase

The oil-in-water emulsion of the present invention comprises a dispersed oil phase.

The amount of oil phase in the emulsion of the invention generally ranges from 3 to 50 wt %, and preferably ranges from 5 to 30 wt % (by total weight oil phase based on the total weight of the emulsion).

The dispersed oil phase may generally be formed from any physiologically acceptable lipophilic material.

Lipophilic materials suitable for use as oil phase components in the invention include both natural and synthetically produced oils, fats and waxes.

Preferred lipophilic materials for use as oil phase components in the invention generally have a liquid or semi-solid consistency at 25° C.

Specific examples of suitable oil phase components include:

oily or waxy hydrocarbons of synthetic, animal or mineral origin: such as mineral oil, petrolatum, paraffin oils such as isoparaffin, ceresin, ozokerite, squalane, squalene, microcrystalline wax, polyethylene wax, polybutene, polyisobutene, and hydrogenated polyisobutene;
silicones: such as dimethicone, dimethicone copolyol, stearoxy dimethicone, silicone wax and cyclomethicone;
higher fatty acids having 6 to 50, preferably 10 to 20, carbon atoms in a molecule: such as isostearic acid, oleic acid, hexanoic acid and heptanoic acid;
fatty alkyl or alkenyl esters having 6 to 50, preferably 10 to 50, carbon atoms in a molecule: such as cetyl 2-ethylhexanoate, cetyl palmitate, C₁₂₋₁₅ alkyl benzoate, octyl palm itate, octyl hydroxystearate, octyldodecyl myristate, octyldodecyl oleate, decyl oleate, stearyl heptanoate, diisostearyl malate, isopropyl linoleate, isopropyl myristate, isopropyl isostearate, isopropyl palmitate, isocetyl stearate, myristyl myristate, myristyl lactate and propylene glycol dicaprylate/dicaprate;
aliphatic higher alcohols having 6 to 50, preferably 10 to 20 carbon atoms in a molecule: such as cetyl alcohol, stearyl alcohol, isostearyl alcohol and oleyl alcohol;
oily, fatty or waxy esters of natural (plant or animal) origin: such as apricot kernel oil, avocado oil, sweet almond oil, beeswax, castor oil, cocoa butter, lanolin, candelilla wax, carnauba wax, shea butter, shea oil, cereal germ oils, cottonseed oil, corn oil, jojoba oil, safflower oil, sunflower oil, olive oil, rapeseed oil, soybean oil, palm kernel oil, babassu kernel oil, coconut oil and medium-chain triglyceride (MCT) oils, which may generally be defined as mixtures of medium chain saturated fatty acids ranging from caproic to lauric (C₆ to C₁₂), in their triglyceride form, and which are typically obtainable from the fractionation of coconut oil.

Particularly good results have been observed with oil-in-water emulsions according to the invention in which the oil phase comprises one or more triglyceride oils. Suitable triglyceride oils are generally naturally derived and include castor oil, caprylic/capric triglycerides, hydrogenated vegetable oil, sweet almond oil, wheat germ oil, sesame oil, hydrogenated cottonseed oil, coconut oil, wheat germ glycerides, avocado oil, corn oil, trilaurin, hydrogenated castor oil, shea butter, cocoa butter, soybean oil, mink oil, sunflower oil, safflower oil, macadamia nut oil, olive oil, apricot kernel oil, hazelnut oil and borage oil.

Mixtures of any of the above described materials may also be used.

### Optional Ingredients

The oil-in-water emulsion of the present invention may advantageously be formulated into a cosmetic or personal care composition, such as a skin or hair care composition. Such compositions will generally contain further ingredients to enhance performance and/or consumer acceptability.

Accordingly, other ingredients typically found in cosmetic or personal care compositions may be added to the oil-in-water emulsion according to the invention.

For example, the dispersed oil phase may also include fragrances, oil-soluble dyes or pigments, lipophilic sun filters, antioxidants, preservatives, and other lipophilic active elements such as lipophilic vitamins and ceramides.

Similarly, the aqueous continuous phase may also include fragrances, water-soluble dyes, preservatives, trace elements, electrolytes (e.g. NaCl or MgSO₄) and other hydrophilic active elements such as hydrophilic sun filters, plant extracts, bacterial extracts, proteins or their hydrolysates (e.g. elastin or collagen hydrolysates), and moisturizers such as polyols. Such polyols represent a preferred class of ingredient for inclusion in the aqueous continuous phase. Suitable examples include glycerol, propylene glycol, 1,3-butylene glycol, sorbitol, hexylene glycol and polymeric polyols such as polypropylene glycol and polyethylene glycol.

The above optional ingredients will generally be present individually in an amount ranging from 0 to 5% by weight individual ingredient based on the total weight of the emulsion.

### Process

The oil-in-water emulsions of the present invention may suitably be prepared by a process comprising the steps of:
(i) dispersing the modified cellulose biopolymer in water under high shear to hydrate it, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
(ii) separately preparing the oil phase, and
(iii) emulsifying the oil phase with an aqueous continuous phase comprising the dispersion obtained in (i), optionally in the presence of a nonionic emulsifier.

As described above, the emulsion so obtained comprises less than 0.2 wt % anionic surfactant (by total weight anionic surfactant based on the total weight of the emulsion).

Nonionic emulsifiers as described above are a preferred ingredient in the emulsions of the invention and are generally incorporated with the oil phase ingredients prepared in step (ii).

Typically in step (iii) the oil phase is added to the aqueous phase, the phases agitated to form a mixture and the resultant mixture is subjected to a mechanical emulsification treatment, thereby forming an oil-in-water emulsion.

The mechanical emulsification treatment may suitably be carried out using high shear mixing or homogenizing equipment known to those skilled in the art, such as a Silverson® mixer or a Microfluidizer®.

Heating may be employed if necessary to aid processing during any or all of the process steps described above.

### Uses

The oil-in-water emulsions of the present invention may suitably be used in cosmetics and dermatology for face creams, body creams, or scalp and hair creams, for cleansing lotions, or body or hair lotions. The emulsions can also be used in makeup products after the addition of pigments such as in mascaras, foundations, and eye liners.

The invention is further illustrated with reference to the following, non-limiting examples. All concentrations are expressed by weight percent of the total formulation, and as level of active matter.

### EXAMPLES

### Example 1

The following formulation represents an emulsion according to the invention.

| Phase | Ingredient | (wt %) |
|---|---|---|
| | Oxidised cellulose⁽¹⁾ | 2 |
| | Tetrasodium EDTA | 0.02 |
| | Neolone ® PE⁽²⁾ | 0.6 |
| A | Sweet almond oil | 5 |
| A | Cetyl ethylhexanoate | 3 |
| A | Miglyol ® 812⁽³⁾ | 2 |
| A | SiClone ® SR5⁽⁴⁾ | 2 |
| A | Emulpharma ® IXL⁽⁵⁾ | 3 |
| A | Cetyl alcohol | 4 |
| | Purified water | q.s. |

⁽¹⁾Partially and selectively oxidised cellulose as described in WO2010/076292
⁽²⁾Methylisothiazolinone (and) phenoxyethanol, ex Dow
⁽³⁾Caprylic/capric triglycerides, ex Sasol
⁽⁴⁾C13-C16 Isoparaffin (and) C12-C14 isoparaffin (and) C13-C15 alkane, ex Presperse, LLC
⁽⁵⁾Ceteth-2, ceteareth-25, lauryl alcohol, cyclopentasiloxane, myristyl alcohol ex Res Pharma

### Method of Manufacture

1) 80 wt % of an aqueous solution of the oxidised cellulose⁽¹⁾ (2.5 wt % a.i.) is added to a main vessel and the remaining water added with continuous stirring.
2) The Tetrasodium EDTA is slowly added with stirring and mixed until fully dispersed.
3) The aqueous phase so obtained is heated to 75° C.
4) In a separate vessel, the Phase A ingredients are mixed and heated to 75° C. until melted and uniform.
5) The main vessel (aqueous phase) is transferred to a Silverson homogeniser and Phase A is slowly added to the main vessel with high shear mixing.
6) The mixture so obtained is emulsified with homogenisation for 5 minutes until bright white and shiny.
7) The mixture is transferred to a paddle stirrer and cooled to below 35° C. with continuous stirring.
8) The Neolone® PE⁽²⁾ is slowly added with continuing stirring and the batch made to weight with purified water.

The emulsion so obtained has a pH of 5.84 and a viscosity of about 30,400 mPa·s (Brookfield RVT Viscometer, Spindle 7, 2.5 rpm, measured after 30 seconds). It is suitable for use as a premium facial skin crème.

The inclusion of the oxidised cellulose⁽¹⁾ was observed to enhance viscosity, structure, skin feel and gloss of the emulsion, compared to a control without this ingredient.

### Example 2

The following formulation represents an emulsion according to the invention.

| Phase | Ingredient | (wt %) |
|---|---|---|
| | Oxidised cellulose⁽¹⁾ | 1 |
| A | Tegocare ® 450⁽⁶⁾ | 3 |
| | Sodium phytate | 0.05 |
| A | Sweet almond oil | 3 |
| A | Sunflower oil | 8 |
| A | Cetyl alcohol | 3 |
| A | Shea butter | 1 |
| | Glycerine | 2 |
| | Curry leaf extract | 0.8 |
| | Purified water | q.s. |

⁽⁶⁾Polyglyceryl-3 methyl glucose distearate, ex Evonik Goldschmidt GmbH

### Method of Manufacture

1) 40 wt % of an aqueous solution of the oxidised cellulose⁽¹⁾ (2.5 wt % a.i.) is added to a main vessel and the remaining water added with continuous stirring
2) The sodium phytate and glycerine are slowly added with stirring and mixed until fully dispersed
3) The aqueous phase so obtained is heated to 75° C.
4) In a separate vessel, the Phase A ingredients are mixed and heated to 75° C. until melted and uniform
5) The main vessel (aqueous phase) is transferred to a Silverson homogeniser and Phase A is slowly added to the main vessel with high shear mixing
6) The mixture so obtained is emulsified with homogenisation for 5 minutes until bright white and shiny
7) The mixture is transferred to a paddle stirrer and cooled to below 35° C. with continuous stirring
8) The extract is slowly added with continuing stirring and the batch made to weight with purified water

The emulsion so obtained has a pH of 5.41 and a viscosity of about 28,400 mPa·s (Brookfield RVT Viscometer, Spindle 7, 2.5 rpm, measured after 30 seconds). It is suitable for use as a 100% natural skin crème.

The inclusion of the oxidised cellulose⁽¹⁾ was observed to enhance stability of the emulsion, compared to a control without this ingredient (in which separation of the emulsion was observed). The oxidised cellulose⁽¹⁾ also produces a bright shiny emulsion with a pleasant skin feel.

To further evaluate the attributes of the oxidised cellulose⁽¹⁾ in emulsions, variants of Examples 1 and 2 above were prepared in which the oxidised cellulose⁽¹⁾ was substituted by xanthan gum or hydroxyethylcellulose respectively, at the same levels. In both cases, the emulsions produced were observed to have inferior appearance and skin feel when compared to the Example 1 and Example 2 formulations.

### Example 3

The following formulation represents an emulsion according to the invention.

| Phase | Ingredient | (wt %) |
|---|---|---|
| | Oxidised cellulose⁽¹⁾ | 2 |
| | Sodium phytate | 0.05 |
| | Betaine | 2 |
| A | Montanov ® 202⁽⁷⁾ | 3 |
| A | Sweet almond oil | 2 |
| A | Sunflower oil | 13 |
| A | Jojoba oil | 2 |
| A | Shea butter | 1 |
| | Rosemary extract | 1 |
| | Curry leaf extract | 1 |
| | Purified water | q.s. |

⁽⁷⁾Arachidyl alcohol, behenyl alcohol, arachidyl glucoside, ex Seppic

### Method of Manufacture

1) 50 wt % of an aqueous solution of the oxidised cellulose⁽¹⁾ (4 wt % a.i.) is added to a main vessel
2) The sodium phytate and betaines are pre-dissolved in the remaining water and this solution added to the main vessel
3) The mixture so obtained is homogenised for 20 minutes and heated to 70° C.
4) In a separate vessel, the Phase A ingredients are mixed and heated to 75° C. until melted and uniform
5) The main vessel (aqueous phase) is transferred to a Silverson homogeniser and Phase A is slowly added to the main vessel with high shear mixing
6) The mixture so obtained is emulsified with homogenisation for 5 minutes until bright white and shiny
7) The mixture is transferred to a paddle stirrer and cooled to below 35° C. with continuous stirring
8) The extracts are slowly added with continuing stirring and the batch made to weight with purified water

The emulsion so obtained has a pH of 5.53 and a viscosity of about 29,500 mPa·s (Brookfield RVT Viscometer, Spindle 7, 2.5 rpm, measured after 30 seconds). It is suitable for use as a 100% natural skin crème.

The inclusion of the oxidised cellulose⁽¹⁾ was observed to help reduce the heaviness and greasiness associated with the use of high levels of vegetable oils.

### Example 4

The following formulation represents an emulsion according to the invention.

| Phase | Ingredient | (wt %) |
|---|---|---|
| | Oxidised cellulose⁽¹⁾ | 2 |
| A | Tegocare ® 450⁽⁶⁾ | 3 |
| A | C₁₂₋₁₅ alkyl benzoate | 12 |
| A | Ethylhexyl methoxycinnamate | 3 |
| A | Butyl methoxydibenzoylmethane | 3 |
| | Methylisothiazolinone | 0.6 |
| | Purified water | q.s. |

### Method of Manufacture

1) 50 wt % of an aqueous solution of the oxidised cellulose⁽¹⁾ (4 wt % a.i.) is added to a main vessel
2) The remaining water is added and the mixture so obtained is homogenised for 20 minutes and heated to 70° C.
3) In a separate vessel, the Phase A ingredients are mixed and heated to 75° C. until melted and uniform
4) The main vessel (aqueous phase) is transferred to a Silverson homogeniser and Phase A is slowly added to the main vessel with high shear mixing
5) The mixture so obtained is emulsified with homogenisation for 5 minutes until bright white and shiny
6) The mixture is transferred to a paddle stirrer and cooled to below 35° C. with continuous stirring
7) The preservative is added with continuing stirring and the batch made to weight with purified water

The emulsion so obtained has a pH of 7.23 and a viscosity of about 23,600 mPa·s (Brookfield RVT Viscometer, Spindle 7, 2.5 rpm, measured after 30 seconds). It is suitable for use as an SPF15 sun protection lotion.

The oxidised cellulose⁽¹⁾ is compatible with UV filters (unlike many conventional hydrocolloids). It provides suitable tactile and film-forming properties without the necessity for additional sensory aids. It also spreads well on the skin and removes the “drag” caused by UV filters. It also provides a pleasant moisturized skin feel without the necessity for additional humectants. In this way the formulation can be significantly simplified compared with conventional sun lotion formulations.

### Example 5

The following formulation represents an emulsion according to the invention.

| Phase | Ingredient | (wt %) |
|---|---|---|
| | Oxidised cellulose⁽¹⁾ | 1.5 |
| A | Tegocare ® 450⁽⁶⁾ | 1.5 |
| A | Cetyl alcohol | 0.3 |
| A | C₁₂₋₁₅ alkyl benzoate | 12 |
| A | Ethylhexyl methoxycinnamate | 3 |
| A | Butyl methoxydibenzoylmethane | 3 |
| | Denatured ethanol | 5 |
| | Methylisothiazolinone | 0.6 |
| | Purified water | q.s. |

### Method of Manufacture

1) 37.5 wt % of an aqueous solution of the oxidised cellulose⁽¹⁾ (4 wt % a.i.) is added to a main vessel
2) The remaining water is added and the mixture so obtained is homogenised for 20 minutes and heated to 70° C.
3) In a separate vessel, the Phase A ingredients are mixed and heated to 75° C. until melted and uniform
4) The main vessel (aqueous phase) is transferred to a Silverson homogeniser and Phase A is slowly added to the main vessel with high shear mixing
5) The mixture so obtained is emulsified with homogenisation for 5 minutes until bright white and shiny
6) The mixture is transferred to a paddle stirrer and cooled to below 35° C. with continuous stirring
7) The ethanol and preservative are added with continuing stirring and the batch made to weight with purified water

The emulsion so obtained has a pH of 7.31 and a viscosity of about 18,400 mPa·s (Brookfield RVT Viscometer, Spindle 7, 2.5 rpm, measured after 30 seconds). It is suitable for use as an SPF15 sun protection spray.

The oxidised cellulose⁽¹⁾ provides pseudoplastic properties so the formulation can be sprayed as a “mist” that reforms on the skin surface.

Furthermore, product stability is maintained at lower viscosities. Therefore the formulator is able to produce sun lotions (e.g. Example 4) or sprays (e.g. Example 5) from essentially the same formulation “chassis”, simply by adjusting the viscosity through the level of oxidised cellulose⁽¹⁾.

### Example 6

The following formulation represents an emulsion according to the invention.

| Ingredient | (wt %) |
|---|---|
| Oxidised cellulose⁽¹⁾ | 2 |
| Betaine | 2 |
| Polyglyceryl-3 cocoate | 3 |
| Ethyl macadamiate (ester of macadamia nut oil) | 2 |
| Chlorella vulgaris extract | 2 |
| Matrixyl ® (peptide active, ex Sederma) | 0.5 |
| Methylisothiazolinone | 0.6 |
| Purified water | q.s. |

### Method of Manufacture

1) 50 wt % of an aqueous solution of the oxidised cellulose⁽¹⁾ (4 wt % a.i.) is added to a main vessel
2) The betaines, chlorella extract and Matrixyl® are added and dispersed into the remaining water. This dispersion is added to the main vessel and the mixture so obtained is homogenised for 20 minutes
3) The polyglyceryl-3 cocoate is added with stirring, then the ethyl macadamiate and the preservative are added and the mixture stirred until uniform
4) The mixture is transferred to a homogeniser and sheared until smooth and shiny

The emulsion so obtained has a pH of 5.56 and a viscosity of about 25,100 mPa·s (Brookfield RVT Viscometer, Spindle 7, 2.5 rpm, measured after 30 seconds). It is suitable for use as a intensive skin treatment serum (e.g. for the eye contour).

The inclusion of the oxidised cellulose⁽¹⁾ was observed to provide a gel-like structure with silky skin feel. Furthermore it is not necessary to include high levels of silicones, which are normally present in conventional intensive skin treatment serums.

### Example 7

The following formulation represents an emulsion according to the invention.

| Phase | Ingredient | (wt %) |
|---|---|---|
| | Purified water | q.s. |
| | Glycerin | 3.5 |
| | Oxidised cellulose⁽¹⁾ | 1.5 |
| | Sodium PCA | 0.2 |
| A | Tegocare ® 450⁽⁶⁾ | 1.5 |
| A | Cetyl alcohol | 0.2 |
| A | Isopropyl myristate | 7 |
| A | Glyceryl monostearate | 1.5 |
| A | Ethylhexyl methoxycinnamate | 1.25 |
| A | Butyl methoxydibenzoylmethane | 0.4 |
| A | Soybean sterol | 0.02 |
| A | Lecithin | 0.01 |
| A | Micronised titanium dioxide | 0.2 |
| A | Niacinamide | 3 |
| A | Milk yoghurt | 0.01 |
| | L-glutamic acid | 0.2 |
| | Methylisothiazolinone, phenoxyethanol | 0.6 |

### Method of Manufacture

1) 37.5 wt % of an aqueous solution of the oxidised cellulose⁽¹⁾ (4 wt % a.i.) is added to a main vessel
2) The remaining water, glycerine and sodium PCA are added and the resulting mixture homogenised for 20 minutes and heated to 70° C.
3) In a separate vessel, the Phase A ingredients are mixed and heated to 75° C. until melted and uniform
4) The main vessel (aqueous phase) is transferred to a Silverson homogeniser and Phase A is slowly added to the main vessel with high shear mixing
5) The mixture so obtained is emulsified until bright and shiny
6) The mixture is transferred to a paddle stirrer and cooled to below 35° C. with continuous stirring
7) The remaining active ingredients and preservative are added with continuing stirring and the batch made to weight with purified water

The emulsion so obtained has a pH of 5.91 and a viscosity of about 18,300 mPa·s (Brookfield RVT Viscometer, Spindle 7, 2.5 rpm, measured after 30 seconds). It is suitable for use as a skin lotion.

The formulation has good skin feel attributes, appearance and tactile properties despite the absence of silicone and paraffin, which are normally present in conventional lotions of this type.

Also, simply by increasing the level of oxidised cellulose⁽¹⁾ in the above formulation to 2 wt %, the same formulation can have the body and consistency of a day cream.

1. An oil-in-water emulsion suitable for cosmetic or personal care use, the emulsion comprising:
a) a aqueous continuous phase;
b) a dispersed oil phase, and
c) optionally, a nonionic emulsifier;
in which the aqueous continuous phase is structured by a dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
and in which the emulsion comprises less than 0.2 wt % anionic surfactant (by total weight anionic surfactant based on the total weight of the emulsion).

2. An oil-in-water emulsion according to claim 1, which comprises from 1 to 5 wt % nonionic emulsifier (by total weight nonionic emulsifier based on the total weight of the emulsion).

3. An oil-in-water emulsion according to claim 2, in which the nonionic emulsifier is selected from fatty acid esters, ethers, hemi-acetals or acetals of polyhydroxylic compounds or a fatty acid amide which is N-substituted with the residue of a polyhydroxylic compound, and mixtures thereof.

4. An oil-in-water emulsion according to claim 1, in which the dispersed oil phase comprises one or more triglyceride oils.

5. A process for preparing an oil-in-water emulsion according to claim 1, comprising the steps of:
(i) dispersing the modified cellulose biopolymer in water under high shear to hydrate it, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
(ii) separately preparing the oil phase, and
(iii) emulsifying the oil phase with an aqueous continuous phase comprising the dispersion obtained in (i), optionally in the presence of a nonionic emulsifier,
in which the emulsion so obtained comprises less than 0.2 wt % anionic surfactant (by total weight anionic surfactant based on the total weight of the emulsion).
